(19) United States
(12) Patent Application Publication
(10) Pub. No.: US 2024/0327769 A1
(43) Pub. Date: Oct. 3, 2024

(54) Organic Solution Production System

(71) Applicants: Nathan Lee Bertsch, Leawood, KS (US); James Patrick Ordo, Avon, IN (US)

(72) Inventors: Nathan Lee Bertsch, Leawood, KS (US); James Patrick Ordo, Avon, IN (US)

(21) Appl. No.: 18/620,409

(22) Filed: Mar. 28, 2024

Related U.S. Application Data
(60) Provisional application No. 63/455,383, filed on Mar. 29, 2023.

(51) Int. Cl. C12M 1/00; A01C 21/00; A01C 23/00; A01C 23/04; C05F 5/00; C12M 1/02; C12M 1/36

(52) U.S. Cl. CPC C12M 23/52; A01C 21/007; A01C 23/042; C05F 5/00; C12M 27/00; C12M 41/14; C12M 41/48; C12M 43/00; A01C 23/008

(57) ABSTRACT

An organic solution production system is provided.

### RELATED APPLICATIONS

This application claims the benefit of U.S. Provisional Patent Application No. 63/455,383 titled “Organic Solution Production System”, to Nathan Lee Bertsch and James Patrick Ordo, filed Mar. 29, 2023, the entire disclosure of which is expressly incorporated by reference herein.

### TECHNICAL FIELD

The present disclosure generally relates to a portable production system to create microbe rich organic solutions to increase agricultural yields.

### BACKGROUND AND SUMMARY OF THE DISCLOSURE

This section introduces aspects that may help facilitate a better understanding of the disclosure. Accordingly, these statements are to be read in this light and are not to be understood as admissions about what is or is not prior art.

In the field of agriculture, there are known benefits of applying a microbe-rich organic solution to crops which include:
Increasing crop yields by activating micro-nutrients in the soil,
Improving soil moisture retention which reduces run-off,
Increasing the resistance of the crop to disease and insect damage, and/or
Supports reduction in the use of chemical fertilizers and pesticides.

In the past, the application of organic solutions with beneficial bacteria, microbes and enzymes has been limited to small-scale hobby farms or mini farms which would apply manure, compost or other organic material to the soil to enhance crop production. Given the success of applying these microbe-rich organic materials to enhance the crop yields, large manufacturers have started production of microbe-rich organic solutions to enhance crop yields and provide other benefits to the farming operation.

Currently, companies formulate organic solutions in large factories and then transport the solution in a concentrated liquid form to farms for application to the crops. This process requires moving containers of concentrated liquid solutions to farms and then mixing with water to allow use of farm sprayers or other means to apply the liquid solution to the fields.

Existing approach introduces several challenges for the farmer. The current organic solution products have limited life after manufacturing and a requirement to control storage temperature prior to application. Because of these limitations, typically farmers only apply a single application of the solution at the start of the growing season. In addition, weather conditions and storage life limitations can impact the timing of the application and result in an application creating less than expected benefits to the crop and soil. The storage life and storage temperature requirements add complexity to the farming operation and prevent achieving the maximum benefit from application of these organic solutions to crops.

According to the present disclosure, the portable solution production system allows production of organic solutions at or near the farm location where the solution will be applied. The production system permits the farm owner to produce multiple batches of an organic solution of beneficial enzymes throughout the growing season to maximize the benefits of the organic solution to the crop. These benefits of these organic solution applications include: improving the effectiveness of traditional chemical fertilizers by improving nutrient absorption from the soil, potential to reduce or eliminate traditional chemical fertilizer applications, potential to reduce or eliminate the use of pesticides and insecticides, and improving the moisture retention in the soil. In addition, the system produces the organic solution in batches which can be directly applied to the soil without need for long term storage.

The portable, self-contained system is portable and is scalable to allow application to farms of various sizes. The scalability of the system allows use of single or multiple organic production systems to be utilized for a single large farm or unit may be scaled to support multiple farms in a farm community.

According to one aspect of the present disclosure, a method for delivering organic solution is provided. The method comprises the steps of providing a portable organic solution production system, transporting the portable organic solution production system to production site, producing organic solution at the production site by mixing measured amounts of raw organic material with water in a mixing tank of the portable organic solution production system and producing microbes in the mixing tank, and delivering the organic solution to a crop application system.

According to another aspect of the present disclosure, an organic solution production system is provided. The organic production system comprises a skid, at least one raw material tank supported by the skid, and at least one mixing tank supported by the skid positioned to receive raw materials from the at least one raw material tank and receive water from a water source.

According to another aspect of the present disclosure, an organic solution production and application system is provided. The organic solution production and application system comprises an organic solution production system including at least one raw material tank, at least one mixing tank configured to produce an organic solution including at least one of microbes and enzymes beneficial to crop production from raw materials from the at least one raw material tank and receive water from a water source, and a controller configured to control the production of the organic solution, and a pivot irrigation system in fluid communication with the organic production system to deliver organic solution to a field.

In addition, system is adjustable to allow for custom formulations based on crop type, soil quality, or other parameters needed to optimize the crop yields. This system is designed to support application to the crops by spraying, pivot irrigation or other methods of application.

### BRIEF DESCRIPTION OF THE DRAWINGS

The previously described aspects of this disclosure will grow to be appreciated at a greater level once references to the following accompanying illustrations are expounded upon.

FIG. 1 is a diagram of an organic solution production system which identifies all the major components and sub-systems. The production system is a portable, self-contained and temperature-controlled unit configured to be used to allow on-site production of organic rich solution for application to fields. The system includes tank(s) for storage of raw materials, along with various pumps, a mixing tank and controls needed to allow production of multiple batches of organic rich solutions during the growing season. A controller is used to monitor and control the production of organic solution and allows fully automated operation including integration with the application processes. The system is designed to support integration to a plurality of application methods.

FIG. 2 shows one embodiment which integrates the production organic solution system to a standard pivot irrigation system. This method allows automation of the production and application of multiple batches of the organic solution to a large field using the pivot irrigation method of application. The field is divided into sectors or zones for applications of the rich organic solution. The organic solution production can be located in the field at a production. This method allows production and application of multiple batches throughout the growing season and the controller permits automation of the application based on a schedule.

The embodiments disclosed below are not intended to be exhaustive or limit the disclosure to the precise form disclosed in the following detailed description. Rather, the embodiments are chosen and described so that others skilled in the art may utilize their teachings. Unless otherwise indicated, the components shown in the figures are shown proportional to each other. It will be understood that no limitation of the scope of the disclosure is thereby intended.

The disclosure includes any alterations and further modifications in the illustrative devices and described methods and further applications of the principles of the disclosure which would normally occur to one skilled in the art to which the disclosure relates.

### DETAILED DESCRIPTION

For the purpose of promoting an understanding of the principles of the present dis closure, reference will now be made to the embodiments illustrated in the figures, and specific language will be used to describe the same. It will nevertheless be understood that no limitation of the scope of this disclosure is thereby intended.

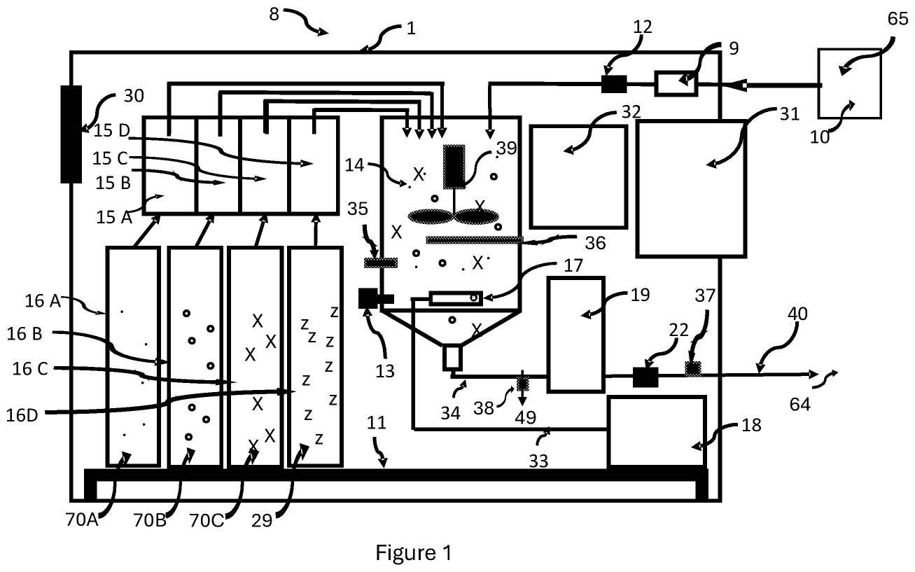

An organic solution production system 8 may consist of the components and sub-systems as shown in FIG. 1. In addition, production system 8 provides a method to produce and apply an organic solution 64 to a farm field(s) 61. Production system 8 is self-contained, portable, scalable and allows installation on or near a farming site. The on-site production permits multiple applications during the year to maximize benefits of organic solution 64 to the crop and soil.

The schematic of the system is shown in FIG. 1. Organic solution 64 production system 8 includes a skid 11 which supports a mixing tank 14 which may be installed into a weatherproof environmentally controlled container 1.

Environmentally controlled container 1 which has mixing tank 14 positioned in the environmentally controlled container 1.

Skid 11 supports mixing tank 14 and the associated hardware provides the necessary elements of the system for production of organic solution 64. Skid 11 provides a structure to mount all major components of organic solution production system 8. Skid 11 allows system to be completely integrated prior to installation into container 1. This approach simplifies assembly, build, and testing phases and simplifies service once the system has been installed in the field.

The production system uses a set of liquified raw organic material(s) 70 (70A, 70B, 70C, etc) stored in one or more raw material tanks 16 (16A, 16B, 16C, 16D, etc). Raw materials in storage tank(s) 16 include organic materials such as molasses, worm castings or other nutrient rich materials which combine to form an organic microbe rich solution. In this embodiment, these raw materials are stored in a liquid form which are combined in mixing tank 14 to create an organic solution 64 that is beneficial to crop production. Beneficial microbes include bacteria which produce enzymes necessary to improve the absorption of micro-nutrients in the soil. The enzymes serve as a catalyst for this process.

Each storage tank(s) 16 has a communication path to a peristaltic pump(s) 15 (15A, 15B, 15C, 15D, etc) or other type of pump(s) which supplies a measured quantity of raw material(s) 70 from storage tank 16 (s) to enter mixing tank 14. A controller 32 provides commands and feedback to deliver a metered quantity of raw material(s) 70 from raw material storage tank(s) 16 to be introduced into mixing tank 14.

An external water source 10 is connected to a valve 9 and a flow meter 12 to provide a measured volume of water 65 to mixing/breeder tank 14 during organic solution 64 production.

Breeder/mixing tank 14 utilizes a tank level sensor 13 and a temperature sensor 35 to monitor the volume and temperature of organic solution 64. Controller 32 controls the production process to create a batch of organic solution 64 by mixing the raw materials and water 65 to produce the desired quantity of organic solution 64.

Aeration of mixing tank 14 is generated by an air compressor 18, which delivers pressurized air to an aerator 17, such as a micro bubbler, via a communication path 33. Aerator 17 is located near the bottom of mixing tank 14 and provides air to promote rapid growth of healthy bacteria. Aeration of organic solution 64 also contributes to mixing the raw materials to achieve the optimum conditions to multiply beneficial microbes. A mechanical mixer 39 is also installed into mixing tank 14 to improve mixing of the raw materials.

A water heater 36 is installed in mixing tank 14 to provide temperature control of organic solution 64 production process to maximize the growth of beneficial bacteria.

Container 1 includes an air vent 30 to allow air to enter and exit the container. Air vent 30 includes an air filter or other method to protect organic production system 1 from airborne contamination such as dust or pollen. In addition, air vent 30 incorporates a heat exchanger or heat recovery ventilator to minimize the energy requirements to maintain temperature control within container 1. An electric heating/cooling sub-system 31 (capable of both heating and cooling) provides a method to maintain the temperature within weatherproof and insulated container 1. Temperature control of container 1 is required to provide an environment which supports long term storage of raw material(s) 70 in tank(s) 16 and supports production of multiple batches of organic solution 64 before it is necessary to refill raw material tank(s) 16.

Production of organic solution 64 and associated growth of healthy bacteria requires approximately 18 to 36 hours to complete the breeding process. During production, controller 32 will control the temperature of breeder tank 14 to maintain a controlled temperature.

The method for delivering organic solution 64 comprising the steps: Organic solution production system 8 is produced in a manufacturing facility where the components can be assembled on skid 11 and integrated into container 1. Once assembled, controller 32 is programmed to control organic solution 64 production process. Organic solution production system 8 unit is then tested at the factory to verify all components are properly integrated. Once an acceptance test is completed, portable organic solution 64 production system 8 is transported to a production site 62.

Once installed at production site 62, organic solution 64 is produced by mixing measured amount of raw organic material(s) 70 with water 65 in a mixing tank 14 of the portable organic solution system 8. Organic solution 64 is produced by using at least one of aerator 17 positioned to provide air bubbles to mixing tank, heating element 36 positioned to provide thermal energy to mixing tank 14 and mixer 39 positioned to mix the raw materials and water 65 in mixing tank 14. Once the organic solution 64 process is completed, the organic solution 64 is delivered to a crop application system, such as a pivot irrigation system 60.

Organic solution production system 8 container 1 is designed to be transportable on a standard flat-bed truck which allows the container to be no larger than 8.5′ tall, 8.5′ wide and less than 53′ long. These dimensions represent the maximum size allowable without specialized permits for oversized truck transportation. System scalability allows for multiple size containers to be produced based on the amount of organic solution 64 production needed to support the farm.

Use of production system container 1 measuring 8′ wide by 8′ long by 8′ tall would allow placement of up to 6 systems on a standard trailer.

Once the container arrives at the farm site, a standard fork truck or other method can be used to place the container on the ground for installation. Once the utilities are connected, and raw materials are added to tank(s) 16, production system 8 will be ready to produce batches of organic solution 64.

When controller 32 starts a production cycle, controller 32 opens valve 9 to allow water 65 to fill tank 14 to a programmed level. Controller 32 commands delivery of raw material from tank(s) 16 to mixing tank 14 by using the pump 15 (s) to flow a measured amount of liquid raw material. Valve 9 opens again to complete filling tank 14 to a level required based on the batch size. The raw materials and batch quantities are all selectable which allows custom batches and formulations based on type of crop, time of year and specific soil conditions.

Once the raw materials are added to mixing tank 14, aeration pump 18 begins to flow air through communication path 33 to aerator 17. Mixer 39 is turned on to ensure a complete mixing of raw organic materials 70 into the water 65. Organic solution 64 batch temperature is monitored by temperature sensor 35 and heater element 36 is used to heat the batch to the optimal temperature to maximize production of beneficial bacteria.

The production process will take approximately 18 to 36 hours to complete. Once a production batch of organic solution 64 is completed, organic solution 64 will be delivered to the distribution system using a communication path 34 to feed a delivery pump 19. Organic solution 64 is pressurized using pump 19 which communicates organic solution 64 from mixing tank to the delivery system via a communication path 40 and a valve 37. Delivery flow rate is measured by a flow meter 22. Delivery pump 19 could be a dosing pump if the delivery method is injection into a pivot style irrigation system or the pump could be centrifugal or other type of pump if the distribution system method uses a tank spray application system. Organic solution 64 may be injected or otherwise provided to crop application systems for application of the organic solution 64 to farms/fields 61. The crop application systems may include field irrigation system 60, mobile tank sprayers, or other application methods.

Once the contents of mixing tank 14 have been transferred to the delivery system and tank 14 is empty, fresh water 65 will be used to flush the mixing tank 14. A biodegradable sanitizing chemical suitable for discharge to the ground at the installation site is added to the water 65 from a tank 16. Sanitizer 29 combined with water 65 is used to flush mixing tank 14 after production of the organic solution 64. Once the flush cycle is initiated, mixing device 39, heater 36, and aerator 17 are used to agitate the flush solution to ensure the breeder tank is sanitized between batches. The flush solution is then drained from mixing tank 14 using communication path 34 to drain flush solution through a valve 38 to a drain 49 which drains flush solution from organic production system 8.

The type and quantities of raw organic material(s) 70 can be adjusted for the specific crop which is being grown. Controller 32 is configured to produce multiple different formulations of batches of organic solution 64 based on different types of crops, soil conditions and timing of the delivery of the organic solution 64 to maximize the benefits to the crop.

The self-contained design allows for placement of one or more system(s) 8 to be installed on individual fields 61 or co-located into a larger facility on a farm 61.

The system is designed to operate either using electrical grid power and/or a renewable energy source such as wind or solar power. Off-grid operation using renewables would require energy storage to allow system to operate continuously. Off-grid electrical power system could also utilize a stand-alone generator as primary power or as back-up to a renewable energy system.

If water source 10 uses a municipal supply, chlorine or chloramines removal will be required. Use of a carbon filter or other pre-treatment will be required as oxidizing chemicals including chlorine and chloramines will prevent the growth of the beneficial microbes needed to produce a healthy organic solution 64.

This approach reduces the volume of transported material and allows for multiple applications to the crops without the requirement to store completed organic solution 64 for long periods of time prior to application.

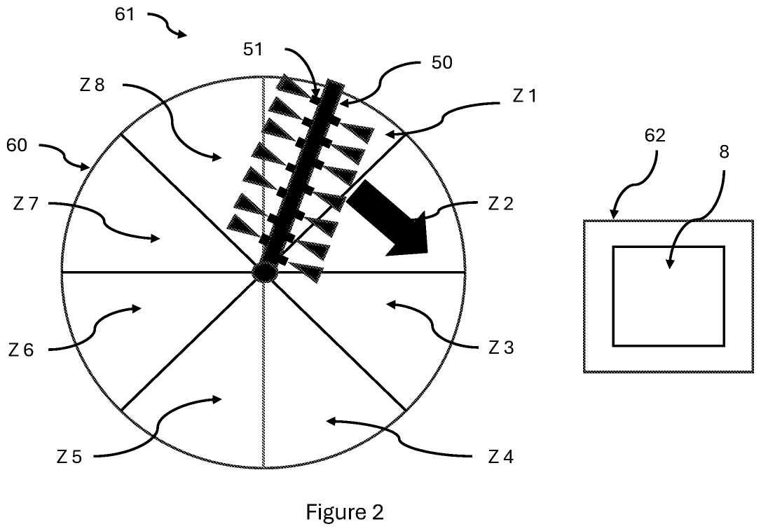

An example of zoning for pivot irrigation system 60 is shown in FIG. 2. FIG. 2 illustrates a field 61 divided into pie shaped zones (Z1, Z2, Z3, Z4, etc.) which allows for integration of a pivot irrigation system 60 in fluid communication with organic solution production system 8 to deliver organic solution 64 to a field. Pivot irrigation system 60 uses a manifold 50 which communicates organic solution 64 mixed with irrigation water to field 61 through a set of nozzles 51 to provide a uniform application of liquid to field 61.

Controller 32 allows programming the delivery of organic solution 64 to each zone(s) Z using the standard irrigation water flow to dilute the concentration of the organic solution 64. Each batch of the organic solution 64 is applied to a separate zone until all zones have received an application of organic solution 64. Controller 32 is configurable to produce multiple batches of organic solution 64 throughout the growing season. In addition, wherein field 61 includes a plurality of zones, controller 32 is configured to deliver different batches of organic solution 64 to the zones.

As an example, a 200 acre farm using a pivot irrigation system as described in the previous section would require production of approximately 13,300 gallons of organic solution 64 to provide 8 applications over a 120 day growing season. Using a pivot irrigation system with 8 zones as shown in FIG. 2 would require production of 64 batches of organic solution 64 during the growing season. Production of this quantity of organic solution 64 would require approximately 300 gallons of raw organic materials 70 combined with 13,000 gallons of water 65. In this example, the mixing tank size would be approximately 250 gallons and each batch would be approximately 210 gallons.

Other field sizes and crops may require larger or smaller batches of organic solution 64 to be produced so the system design is scalable to generate the required quantity of organic solution 64 for individual farms or a community of farms. System scalability allows large farms comprising a plurality of pivot irrigation systems to utilize a single organic solution production system 8 to deliver organic solution 64 to a plurality of fields 61.

In addition, this system could be used for other plant growing applications in addition to conventional farms including green houses, hydroponics, vertical farming or other farming methods.

The production organic solution system 8 allows complete scalability by sizing raw material storage tanks 16 and mixing tank 14 for a smaller or larger batch size. Raw material storage tanks 16 using standard size containers (30 or 55 gallon) to allow rapid change out without the need to move liquid from tank to tank when containers require re-filling.

Using multiple batch production allows the unit to have a small physical size and create a portable system capable of supplying large quantities of organic solution 64 over a growing season. For the 200 acre example, an 8′ by 8′ by 8′ container which contains 512 cubic foot can produce approximately 13,300 gallons of organic solution 64 during the 120 day growing season.

The system is very compact and permits production of organic solution 64 using a small container 1. For example, the preferred production system uses an 8 foot tall by 8 foot wide by 8 foot long container with a 250 gallon mixing tank, the ratio of container volume to mixing tank volume is approximately 15 to 1.

Controller 32 is used to fully automate the production process which includes thermal management of the container 8 and mixing tank 14, measuring and mixing quantities of the raw material 70 necessary to produce a batch of organic solution 64, aeration of solution, process cycle timers, transfer of production organic solution 64 when completed to the delivery system, tank flushing, and monitoring all critical process variables to ensure consistent production of a batch of organic solution.

In addition, controller 32 will include the ability to monitor process remotely using a communication protocol and provide diagnostic to a remote site including system performance, raw material status, and equipment health status. Controller 32 could also communicate maintenance status and batch parameters needed to verify proper operation without the need to physically visit the farm or location of the portable unit.

The self-contained portable system allows for easy placement or removal and enables system to be easily transported to a remote maintenance location for storage or update once the growing season has ended. This ability to quickly remove the unit from a site provides options to sell or lease units to farm owners. In addition, independent companies could use the capability to produce the organic solution locally to reduce transportation and storage costs.

An alternative approach would be to install one or more equipment skid(s) 11 with all the necessary equipment into a climate-controlled building. This approach may be cost effective if an existing building has adequate floor space and the required utilities to operate the system.

This embodiment shows a unit capable of supporting multiple applications of the organic solution for a 200 acre plot of land. The entire system including sizes of the raw material storage vessels and mixing tank can be scaled to a larger or smaller size to accommodate different size farms or allow differing dosing concentrations or frequency.

The described embodiment is designed to feed a pivot irrigation type delivery system. Other means of delivery could include tanker type sprayers, aerial crop spraying, standard ground irrigation or other means to apply the product to the soil.

According to one embodiment, controller 32 includes maintenance screens to log operational data supplied by one or more sensors 35 are used to control the system. Additional pressure, temperature and flow sensors may be added to provide additional system data for maintenance and remote monitoring of the system. Maintenance screens may display readings supplied by sensors, measure number of batches produced, batch temperature, raw material levels, system faults, including but not limited to low pressure of feed water 65, pump 19 fault, low levels of raw material, etc. According to alternative embodiments, readings displayed by maintenance screens may use remote connection capable to support scheduling of maintenance actions remotely. In addition, controller 32 can include a touch screen to allow operators to interact with a simple intuitive interface. A touch screen interface also facilitates complete tracking of system operation with date stamps and diagnostic codes to provide a full history of the unit operational history.

Those having ordinary skill in the art will recognize that numerous modifications can be made to the specific implementations described above. The implementations should not be limited to the particular limitations described. Other implementations may be possible.

We claim:

1. A method for delivering organic solution comprising the steps of:
providing a portable organic solution production system,
transporting the portable organic solution production system to production site,
producing organic solution at the production site by
mixing measured amounts of raw organic material(s) with water in a mixing tank of the portable organic solution production system and
producing microbes in the mixing tank, and
delivering the organic solution to a crop application system.

2. The method of claim 1, wherein the portable organic solution production system includes a skid supporting the mixing tank.

3. The method of claim 1, wherein the portable organic solution production system includes an environmentally controlled container, and the mixing tank is positioned in the environmentally controlled container.

4. The method of claim 1, wherein the environmentally controlled container includes a container volume and the mixing tank includes a tank volume and a ratio of the container volume to the tank volume is less than 150.

5. The method of claim 4, wherein the ratio is less than 50.

6. The method of claim 5, wherein the ratio is less than 20.

7. Organic solution production system comprising:
a skid,
at least one raw material tank supported by the skid, and
at least one mixing tank supported by the skid positioned to receive raw materials from the at least one raw material tank and receive water from water source.

8. The organic solution production system of claim 7, further comprising at least one of an aerator positioned to provide air bubbles to the mixing tank, a heating element positioned to provide thermal energy to the mixing tank, and a mixer positioned to mix the raw materials and water in the mixing tank.

9. The organic solution production system of claim 7, further comprising a delivery pump which communicates organic solution from the mixing tank to a crop application system.

10. The organic solution production system of claim 7, further comprising a sanitizer in communication with the mixing tank.

11. An organic solution production and application system comprising:
an organic solution production system including
at least one raw material tank,
at least one mixing tank configured to produce an organic solution including at least one of microbes and enzymes beneficial to crop production from raw materials from the at least one raw material tank and receive water from water source, and
a controller configured to control the production of the organic solution, and
a pivot irrigation system in fluid communication with the organic solution production system to deliver organic solution to a field.

12. The organic solution production system of claim 11, wherein the controller is configured to produce multiple batches of organic solution.

13. The organic solution production system of claim 12, wherein the field includes a plurality of zones and the controller is configured to control delivery of multiple batches of the organic solution to the zones.

14. The organic solution production system of claim 12, wherein the controller is configured to produce multiple different formulations of batches of organic solution based on different types of crops, soil conditions, and timing of the delivery of the organic solution.

15. The organic solution production system of claim 11, further comprising a plurality of pivot irrigation systems in fluid communication with the organic production system to deliver organic solution to a plurality of fields.
